(19) United States
(12) Patent Application Publication
(10) Pub. No.: US 2024/0360485 A1
(43) Pub. Date: Oct. 31, 2024

(54) PROCESS FOR PREPARATION OF PHLOROGLUCINOL AND PHENOLIC DERIVATIVES

(71) Applicant: Jeffrey Ehrhardt, San Diego, CA (US)

(72) Inventor: Jeffrey Ehrhardt, San Diego, CA (US)

(21) Appl. No.: 18/651,463

(22) Filed: Apr. 30, 2024

Related U.S. Application Data
(60) Provisional application No. 63/463,058, filed on Apr. 30, 2023.

(51) Int. Cl. C12P 7/22; C12N 9/20; C12P 7/42

(52) U.S. Cl. CPC C12P 7/22; C12N 9/20; C12P 7/42; C12Y 301/01003; C12Y 301/01023; C12Y 307/01004; C12Y 307/01008; C12Y 307/01019

(57) ABSTRACT

Processes for production of phloroglucinol or derivative thereof. An aspect of the present disclosure relates to an enzymatic process for production of phloroglucinol or derivative thereof from polyphenol. The present disclosure also relates to a process for production of phenolic derivatives. Accordingly, another aspect of the present disclosure relates to an enzymatic process for production of phenolic derivatives from polyphenol. The processes of the present disclosure affords facile and environment friendly production of phloroglucinol or derivative(s) thereof and/or phenolic derivative(s).

### CROSS-REFERENCE TO RELATED APPLICATIONS

This application claims the benefit under 35 U.S.C. § 119(e) of U.S. Provisional Patent Application No. 63/463,058, which was filed Apr. 30, 2023 and titled “A PROCESS FOR PREPARATION OF PHLOROGLUCINOL AND PHENOLIC DERIVATIVES,” which is hereby incorporated herein by reference in its entirety.

### FIELD OF THE INVENTION

The present disclosure relates to a process for production of phloroglucinol or derivative thereof. An aspect of the present disclosure relates to an enzymatic process for production of phloroglucinol or derivative thereof from polyphenol. The present disclosure also relates to a process for production of phenolic derivatives. Accordingly, another aspect of the present disclosure relates to an enzymatic process for production of phenolic derivatives from polyphenol.

### BACKGROUND

Phloroglucinol is a colourless solid organic compound having chemical formula C₆H₃(OH)₃. Phloroglucinol and derivatives thereof find application in wide variety of areas such as—as a coupling agent in printing, for synthesis of pharmaceuticals (e.g. Flopropione), in cosmetics and in nutrition. However, production of phloroglucinol is challenging and expensive. The conventional processes for production of phloroglucinol or derivatives thereof make use of benzene as the starting material. Further, such conventional processes for production of phloroglucinol or derivatives thereof involve usage of explosive and toxic intermediates for example, benzene trinitrate (trinitrobenzene).

Accordingly, need is felt of a new and improved process for production of phloroglucinol or derivative thereof that may overcome one or more problems associated with the conventional processes. Need is also felt of a new and improved process for production of phenolic derivatives. The present invention satisfies the existing needs, as well as others, and generally overcomes the deficiencies found in the state of art.

### Objects

An object of the present invention is to provide a new and improved process that may overcome one or more limitations associated with the conventional processes for production of phloroglucinol or derivative thereof.

It is an object of the present disclosure to provide an improved process for production of phloroglucinol or derivative thereof from polyphenol.

It is also an object of the present disclosure to provide an improved process for production of phloroglucinol or derivative thereof from polyphenol that precludes production/usage of explosive and toxic intermediates such as benzene trinitrate (trinitrobenzene).

Another object of the present disclosure is to provide a process for production of phloroglucinol or derivative thereof from polyphenol that is safe and environment friendly.

Further object of the present disclosure is to provide a process for production of phloroglucinol or derivative thereof from polyphenol that is easy to follow, economical and industrially applicable.

Still further object of the present disclosure is to provide a process for production of phenolic derivatives from polyphenol that is easy to follow, economical and industrially applicable.

### SUMMARY

The present disclosure relates to a process for production of phloroglucinol or derivative thereof from polyphenol. An aspect of the present disclosure relates to an enzymatic process for production of phloroglucinol or derivative thereof from polyphenol. The present disclosure also relates to a process for production of phenolic derivatives. Accordingly, another aspect of the present disclosure relates to an enzymatic process for production of phenolic derivative(s) from polyphenol.

An aspect of the present disclosure relates to an enzymatic process for production of phloroglucinol or derivative thereof from polyphenol. The process comprises contacting a polyphenol with an enzyme selected from a hydrolase and a lipase, optionally, at a pH higher than about 8.0, to obtain phloroglucinol or derivative thereof.

Another aspect of the present disclosure relates to an enzymatic process for production of a phenolic derivative of Formula C from polyphenol. The process comprises contacting a polyphenol with an enzyme selected from a hydrolase and a lipase, optionally, at a pH higher than about 8.0, to obtain the phenolic derivative of Formula C,

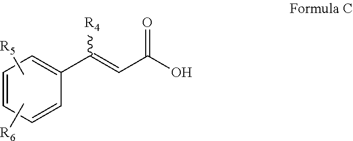

wherein R₄ represents H, OH or —OR7, R₇ being a sugar moiety; and R₅ and R₆ independently represents H, OH, C1-C4 alkyl group or —OR₈, R₈ being a C1-C4 alkyl group or a sugar moiety.

In some embodiments, the polyphenol is represented by Formula A or Formula B,

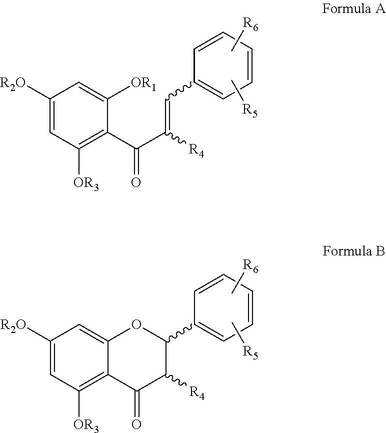

wherein R₁, R₂ and R₃ independently represents H, C1-C4 alkyl group and a sugar moiety; R₄ represents H, OH or —OR7, R₇ being a sugar moiety; R₅ and R₆ independently represents H, OH, C1-C4 alkyl group or —OR₈, R₈ being a C1-C4 alkyl group or a sugar moiety.

Without wishing to be bound by the theory, it is believed that the hydrolase or the lipase enzyme effects hydrolysis of the chalcone resulting in production of phloroglucinol or derivative thereof and a phenolic derivative. The reaction is schematically represented below:

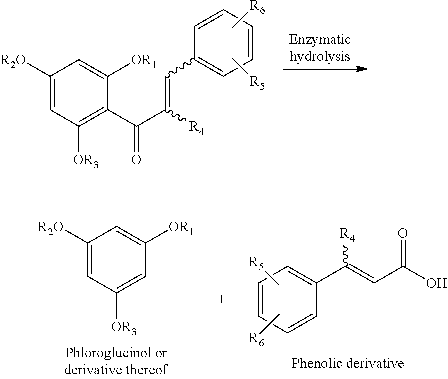

It is also believed, without wishing to be bound by the theory, that when the polyphenol has a 3,4-dihydro-2H-chromene skeleton, for example, as shown in Formula B above (as against the chalcone skeleton, for example, as shown in Formula A above) it is advantageous to contact the polyphenol with the enzyme at a pH higher than about 8.0 (for example, by effecting addition of a base), wherein the basic conditions aid in ring opening affording conversion of 3,4-dihydro-2H-chromene moiety to chalcone moiety, which, as mentioned above, undergoes enzymatic hydrolysis affording facile production of phloroglucinol or derivative thereof and a phenolic derivative. The reaction is schematically represented below:

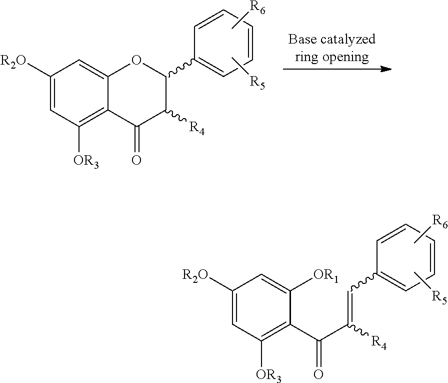

In some embodiments, the polyphenol is selected from flavonoid aglycones, flavonoid glycosides, chalcones and mixtures thereof.

In some embodiments, the polyphenol is selected from naringin, naringenin, hesperidin, hesperetin, eriodictyol, homoeriodictyol, rutin, quercetin, dihydrokaempferol, dihydroquercetin, hesperidin chalcone, hesperetin chalcone, naringin chalcone and mixtures thereof.

In some embodiments, the polyphenol is selected from naringin, naringenin, hesperidin, hesperetin, eriodictyol, homoeriodictyol, rutin, dihydrokaempferol, dihydroquercetin, hesperidin chalcone, hesperetin chalcone, naringin chalcone, liquiritigenin, afzelechin, mesquitol, chalcone, echinatin and mixtures thereof.

Other aspects, advantages, and salient features of the invention will become apparent to those skilled in the art from the following detailed description, which, taken in conjunction with the exemplary embodiments of the invention.

### BRIEF DESCRIPTION OF THE DRAWINGS

FIG. 1 shows HPLC analysis results, demonstrating that hesperetin chalcone is converted to phloroglucinol and isoferulic acid by the hydrolase enzyme.

FIG. 2A shows colorimetric screening assay for phloroglucinol production.

FIGS. 2B and 2C are images showing the results of a 37-enzyme screening panel, with the colorimetric confirmation of hydrolysis of both hesperetin and hesperetin chalcone.

### DETAILED DESCRIPTION

The present invention relates to a process for production of phloroglucinol or derivative thereof from polyphenol. The present disclosure also relates to a process for production of phenolic derivatives.

The following is a detailed description of embodiments of the present invention. The embodiments are in such detail as to clearly communicate the invention. However, the amount of detail offered is not intended to limit the anticipated variations of embodiments; on the contrary, the intention is to cover all modifications, equivalents, and alternatives falling within the spirit and scope of the present invention as defined by the appended claims.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases, it will be recognized that references to the “invention” will refer to subject matter recited in one or more, but not necessarily all, of the claims.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability.

Unless the context requires otherwise, throughout the specification which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

As used in the description herein and throughout the claims that follow, ranges and amounts can be expressed as “about” or “approximately” with respect to a particular value or range of values. The terms “about” or “approximately” also include the exact amount. For example, “about 5 grams” means “about 5 grams” and “5 grams.” In general, the term “about” includes an amount that is expected to be within experimental error.

As used in the description herein and throughout the claims that follow, “about the same” means within an amount that one skilled in the art considers to be the same or to be within an acceptable range of error.

As used in the description herein and throughout the claims that follow, the terms “optional” or “optionally” mean that the subsequently described event or circumstance does or does not occur.

As used in the description herein and throughout the claims that follow, a “buffer” refers to a substance, generally a solution, that can keep its pH constant, despite the addition of strong acids or strong bases and external influences of temperature, pressure, volume, or redox potential. A buffer prevents change in the concentration of another chemical substance, e.g., proton donor and acceptor systems that prevent marked changes in hydrogen ion concentration (pH). The pH values of all buffers are temperature and concentration dependent. The choice of buffer to maintain a pH value or range can be empirically determined by one of skill in the art based on the known buffering capacity of known buffers.

As used in the description herein and throughout the claims that follow, acidic pH are all values from 0 to 7; neutral pH is a value equal to 7, and basic pH are all values from 7 to 14.

As used in the description herein and throughout the claims that follow, “contacting” refers to a process of bringing chemicals and enzymes or catalysts in proximity in solution, gas, or solid phase, such that a chemical interaction, which facilitates the claimed processes, occurs. Contacting may refer to interactions between freely diffusing components in the reaction, interactions between a freely diffusing component and an immobilized enzyme, or solid state interactions between two components.

In some embodiments, the numbers expressing quantities of ingredients, properties such as concentration, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it were individually recited herein.

The headings and abstract of the invention provided herein are for convenience only and do not interpret the scope or meaning of the embodiments.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

An aspect of the present disclosure relates to an enzymatic process for production of phloroglucinol or derivative thereof from polyphenol. The process comprises contacting a polyphenol with an enzyme selected from a hydrolase and a lipase, optionally, at a pH higher than about 8.0 to obtain phloroglucinol or derivative thereof.

Another aspect of the present disclosure relates to an enzymatic process for production of a phenolic derivative of Formula C from polyphenol. The process comprises contacting a polyphenol with an enzyme selected from a hydrolase and a lipase, optionally, at a pH higher than about 8.0, to obtain the phenolic derivative of Formula C,

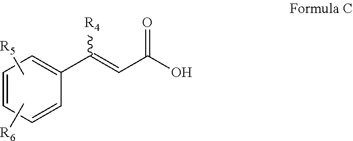

wherein R₄ represents H, OH or —OR7, R₇ being a sugar moiety; and R₅ and R₆ independently represents H, OH, C1-C4 alkyl group or —OR₈, R₈ being a C1-C4 alkyl group or a sugar moiety.

In some embodiments, the polyphenol is represented by Formula A or Formula B,

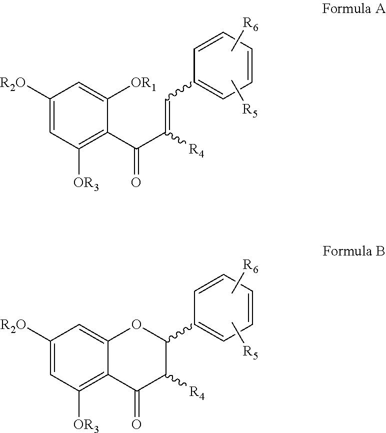

wherein R₁, R₂ and R₃ independently represents H, C1-C4 alkyl group and a sugar moiety; R₄ represents H, OH or —OR7, R₇ being a sugar moiety; R₅ and R₆ independently represents H, OH, C1-C4 alkyl group or —OR₈, R₈ being a C1-C4 alkyl group or a sugar moiety.

In some embodiments, the polyphenol is selected from flavonoid aglycones, flavonoid glycosides, chalcones and mixtures thereof, for example, naringin, naringenin, hesperidin, hesperetin, eriodictyol, homoeriodictyol, rutin, quercetin, dihydrokaempferol, dihydroquercetin, naringenin chalcone, liquiritigenin, afzelechin, mesquitol, chalcone, echinatin, hesperidin chalcone, hesperetin chalcone, naringin chalcone and mixtures thereof.

In some embodiments, the polyphenol is represented by Formula B, and is selected from naringin, naringenin, hesperidin, hesperetin, eriodictyol, homoeriodictyol, rutin, quercetin, dihydrokaempferol, dihydroquercetin and mixtures thereof.

In some embodiments, the polyphenol is represented by Formula A, and is selected from hesperidin chalcone, hesperetin chalcone, naringin chalcone and mixtures thereof.

Without wishing to be bound by the theory, it is believed that the hydrolase or the lipase enzyme effects hydrolysis of the chalcone resulting in production of phloroglucinol or derivative thereof. The reaction is schematically represented below:

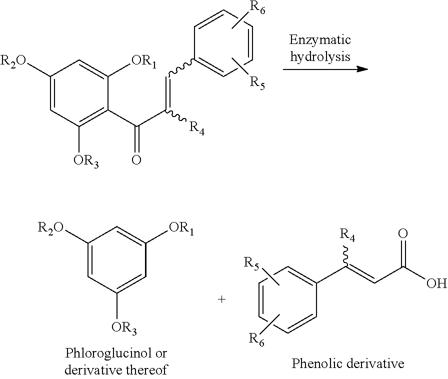

It is also believed, without wishing to be bound by the theory, that when the polyphenol has a 3,4-dihydro-2H-chromene skeleton, for example, as shown in Formula B above (as against the chalcone skeleton, for example, as shown in Formula A above) it is advantageous to contact the polyphenol with the enzyme at a pH higher than about 8.0 (for example, by effecting addition of a base), wherein the basic conditions aid in ring opening affording conversion of 3,4-dihydro-2H-chromene moiety to chalcone moiety, which, as mentioned above, undergoes enzymatic hydrolysis affording facile production of phloroglucinol or derivative thereof and a phenolic derivative. The reaction is schematically represented below:

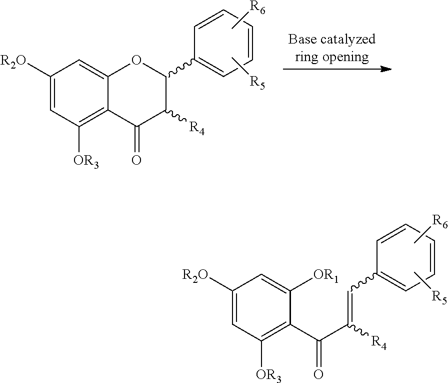

In some embodiments, the polyphenol comprises one or more polyphenols extracted from or otherwise derived from a plant source. In some embodiments, the polyphenol comprises one or more plant extracted polyphenols.

In some embodiments, the polyphenol comprises one or more polyphenols chemically synthesized from petrochemicals.

In some embodiments, the polyphenol comprises one or more polyphenols produced using engineered microbial strains using precision fermentation.

In some embodiments, the polyphenol is contacted with an enzyme in presence of a buffer. Exemplary buffers suitable for the processes of the present disclosure includes, but not limited to, salts of phosphate, carbonate, tricine, bicine, hepes, 3-morpholinopropane-1-sulfonic acid (MOPS), 2-(N-morpholino)ethanesulfonic acid (MES), N-[Tris(hydroxymethyl)methyl]-2-aminoethanesulfonic acid (TES), 3-[Tris-(hydroxymethyl) Methylamino]-2-Hydroxypropane Sulphonic Acid (TAPSO), glycine, lysine, acetate, borate and mixtures thereof. In some embodiments, the buffer comprises an aqueous solution of buffer. The buffer can be of a strength ranging from about 1 mM to about 1000 mM, for example, from about 5 mM to about 750 mM or from about 10 mM to about 500 mM or from about 50 mM to about 500 mM or from about 50 mM to about 300 mM or from about 50 mM to about 250 mM.

In some embodiments, the basic pH is obtained by addition of a base. Exemplary bases suitable for the processes of the present disclosure includes, but not limited to, an inorganic base, an organic base and mixtures thereof, for example, sodium hydroxide, potassium hydroxide, potassium carbonate, sodium carbonate, lithium hydroxide, calcium hydroxide, ammonium hydroxide, pyridine, triethylamine, trimethylamine, aniline, urea, aluminum hydroxide, sodium bicarbonate and mixtures thereof.

Some embodiments herein describe an enzymatic process for production of phloroglucinol and isoferulic acid from hesperetin, comprising contacting hesperetin with a hydrolase enzyme at a basic pH. The basic conditions aid in ring opening affording conversion of hesperetin to hesperetin chalcone, which subsequently undergoes enzymatic hydrolysis affording facile production of phloroglucinol and isoferulic acid.

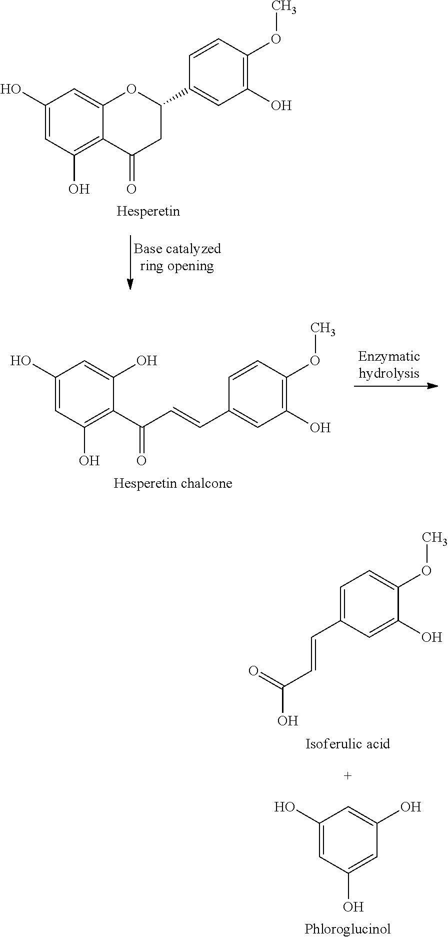 [SMILES: COc1ccc([C@@H]2CC(=O)c3c(O)cc(O)cc3O2)cc1O ; COc1ccc(/C=C/C(=O)c2c(O)cc(O)cc2O)cc1O ; COc1ccc(/C=C/C(=O)O)cc1O ; Oc1cc(O)cc(O)c1]

Exemplary hydrolase enzymes suitable for the processes of the present disclosure includes, but not limited to, 2,6-dihydroxypseudooxynicotine hydrolase (EC No. 3.7.1.19), 2,4-diacetylphloroglucinol hydrolase (EC No. 3.7.1.24), phloretin hydrolase (EC No. 3.7.1.4), and 2,6-dioxo-6-phenylhexa-3-enoate hydrolase (EC No. 3.7.1.8).

Protein sequences corresponding to the hydrolase enzymes are well known in the art and the same can be used in the processes of the present disclosure. Exemplary UNIPROT sequence IDs for PhlG suitable for the processes of the present disclosure include: A0A291T456; A6VR30; C5S4Y7; D6PW75; G8QCB2; Q4JIX4; R7IJR8. Exemplary UNIPROT sequence IDs for Phloretin hydrolase suitable for the processes of the present disclosure include: A0A0B2JWC7; A0A174E3K5; A0A174LIJ7; A0A1B1KQ64; A0A1D3NLN1; A0A1Q5TII4; A0A1Q8RT53; A0A1V9W609; A0A1W6WQN5; A0A1Y3R8X2; A0A1Y3RWU8; A0A1Y3RZ09; A0A1Y3SA13; A0A1Y3SGA0; A0A1Y3XDD5; A0A1Y3ZWB9; A0A1Y4C576; A0A1Y4FKN3; A0A1Y4L4Z4; A0A1Y4LQ02; A0A1Y4UI02; A0A1Y4WD57; A0A221C075; A0A243ICI0; A0A2A2P4R4; A0A2A8U0P6; A0A2A8UQC4; A0A2A8UQH4; A0A2A8UV90; A0A2B1DZ08; A0A2B1KX20; A0A2B2G8E6; A0A2C1LDS0; A0A2C1LJI8; A0A2C1Z647; A0A2C1ZI69; A0A2C1ZIU9; A0A2C2A4M0; A0A2C3D761; A0A2G7ENA7; A0A2I7W7H7; A0A2N6NVX2; A0A2N9Z2I7; A0A2P1TUQ6; A0A2S3U3Z7; A0A2S8VI52; A0A2S9VJH5; A0A2S9VYT1; A0A2V1JL76; A0A2V1JQB9; A0A317VMP2; A0A395ML34; A0A395S8D9; A0A3B9B7K0; A0A3B9B839; A0A3C0KIS2; A0A3D0GR21; A0A3D1YRS9; A0A3D2MTP4; A0A3D3TBV1; A0A3D5MXG4; A0A415E6X5; A0A415R9W5; A0A417VFG7; A0A417VHA0; A0A419T330; A0A420GMX2; A0A443T9N7; A0A484FNA4; A0A4U6XJV2; A0A4V4NCB1; A0A4Z1P6I0; A0A516QUF4; A0A523AWY1; A0A523AYA6; A0A523B1I4; A0A524DJB9; A0A524FCJ6; A0A532TMS2; A0A5M9HZU5; A0A5N5CYU0; A0A5N6D9L9; A0A5N6EW88; A0A5N6WGB4; A0A5N6WV28; A0A5Q4BAM5; A0A6A7K5H1; A0A6N7W1P9; A0A7C2ILM0; A0A7C2IM49; A0A7C5YN73; A0A7C6BT65; A0A7C6FWS7; A0A7G5P178; A0A7J3I4U2; A0A7J3NZI8; A0A7J6JHD7; A0A826H206; A0A843QW11; A0A844DUH8; A0A844DZY7; A0A847WAA0; A0A849EV87; A0A8H4C549; A0A8H4L140; A0A8H4NQ51; A0A8H5KR17; A0A8H5MUL4; A0A8H5PS69; A0A8H5Q605; A0A8H5S8D7; A0A8H5TLH4; A0A8H5YHT7; A0A8J5P5H3; A0A8J6JB28; A0A8J6JEQ8; A0A8J6JFA3; A0A8J6JHS5; A0A8J7VGU7; A0A8J7VKP9; A0A8T6RPQ5; A0A927VJY2; A0A927VKZ6; A0A927Z9P1; A0A928MC47; A0A940TM53; A0A940TSI7; A0A943C4H9; A0A943GYG7; A0A949THA4; A0A970ECS0; A0A970FYM9; A0A970JF29; A0A970K5J1; A0A970KQF3; A0A970QZX9; A0A970U9G7; A0A970XAJ9; A0A970XAT0; A0A970YA82; A0A970YE35; A0A974XGL2; C2XTW7; J4KNR0; L2FPX5; R5TJH4; U5RR55; W6T414. Exemplary UNIPROT IDs for Phloretin hydrolase (EC 3.7.1.4) include: A0A143QDR1; A0A143QLS8; A0A161GDG8; A0A1X0L2V1; A0A231H182; A0A2H6B7R6; A0A2Z4UGV2; A0A375YE64; A0A395L4A3; A0A3G8JHT0; A0A498Q2A6; A0A498Q3Q5; A0A498QX62; A0A5S9N857; A0A6P2R6D9; A0A7K0DAH7; A0A7K0DP09; A0A7Z7IMZ4; Q715L4. Exemplary UNIPROT sequence ID for Phloretin hydrolase (EC 3.7.1.4) (mPhlG) includes B1MK49. Exemplary UNIPROT sequence ID for Phloretin hydrolase protein includes A0A0N0DBM5. Exemplary UNIPROT sequence ID for predicted Phloretin hydrolase includes D8GPY1. Exemplary UNIPROT sequence ID for Putative hydrolase enzyme suitable for the processes of the present disclosure includes B2FTX7. Exemplary UNIPROT sequence ID for Putative phlG protein suitable for the processes of the present disclosure is A0A829QJC8. Exemplary UNIPROT sequence ID for Putative phloretin hydrolase protein suitable for the processes of the present disclosure is R1GJH8. Exemplary UNIPROT sequence IDs for 2,4-diacetylphloroglucinol hydrolase suitable for the processes of the present disclosure include: A0A084D908; A0A086GEN4; A0A0D0NNZ8; A0A0G3GQ96; A0A0P0QF59; A0A0Q7AT34; A0A125SE96; A0A159ZXW2; A0A176NEI5; A0A1E3YC51; A0A1E4W7B1; A0A1V3SEM5; A0A292ACD4; A0A2J7U361; A0A2K4IPP6; A0A2M8SI76; A0A2T6GKS5; A0A2W0FFD9; A0A2W1N416; A0A3M1UVK6; A0A423GB76; A0A4D7Z2T0; A0A4Q5TQ82; A0A546Z3T8; A0A6N1C869; A0A7G7X4N9; A0A7V8EXW0; A0A7X1KEA4; A0A7X1PH13; A0A7X3HDG4; A0A7X5MRG3; A0A7Y1GE60; A0A7Y6QR29; A0A7Y7XY34; A0A7Y8CME4; A0A7Z2THQ1; A0A8F9UVZ3; A0A8I0HRQ4; and F2KGR4. Exemplary UNIPROT sequence IDs for 2,4-diacetylphloroglucinol hydrolase (DAPG hydrolase) (EC 3.7.1.24) suitable for the processes of the present disclosure include: A0A2C9EVE6; A0A2K9M484; F7J5X9; Q4K423. Exemplary UNIPROT sequence IDs for 2,4-diacetylphloroglucinol specific hydrolase PhlG suitable for the processes of the present disclosure include: A0A0D6BR19; A0A1U9JTE5; A0A822VCC5; I4KJG5; J2MRE1; K2Q4U1. Exemplary UNIPROT sequence IDs for DAPG hydrolase PhiG domain protein suitable for the processes of the present disclosure include: A0A853SYS5; A0A853T172. Exemplary UNIPROT sequence IDs for DAPG hydrolase PhiG domain-containing protein suitable for the processes of the present disclosure include: A0A024JZI3; A0A031GLX6; A0A031HU98; A0A045ITU5; A0A064CIE8; A0A077KIX2; A0A084DCI3; A0A096BCZ6; A0A0A0DTL7; A0A0A2HGH6; A0A0A8EY57; A0A0C4YP82; A0A0C4YR39; A0A0C4YUJ7; A0A0D0GSP3; A0A0D1JWJ3; A0A0D5ACL9; A0A0D5BKR5; A0A0D6HQP8; A0A0D6SYV4; A0A0E3JZE6; A0A0E4CNF0; A0A0F5NHW1; A0A0F6W9H7; A0A0G3IDW3; A0A0H2LV18; A0A0H3LAK7; A0A0H3M4P4; A0A0J6VK90; A0A0J6VTZ2; A0A0J6Y8Y5; A0A0K2HW64; A0A0K9GPC6; A0A0L6CP59; A0A0M2H5P5; A0A0M2WPF5; A0A0M7DWU1; A0A0M7MDZ2; A0A0N1NDL0; A0A0N8QAJ1; A0A0P0NYS1; A0A0P9CYS8; A0A0Q0J4L0; A0A0Q2LLT1; A0A0Q2M7M7; A0A0Q5R479; A0A0Q6GN82; A0A0Q6JJQ6; A0A0Q6K7E7; A0A0Q6KLU3; A0A0Q7N9Y0; A0A0R1ZI27; A0A0R2BEW5; A0A0S6U356; A0A0S8D495; A0A0T6Y053; A0A0T9Z6J7; A0A0U0W5F3; A0A0U1DJE7; A0A0U3MPZ2; A0A100WK69; A0A100X6Q6; A0A101A055; A0A109SWZ9; A0A117J8E3; A0A124EQA3; A0A150A661; A0A162E016; A0A162R0A3; A0A164SKG4; A0A166HNF7; A0A172UG98; A0A173LQZ2; A0A173U3M8; A0A173VKL3; A0A174LRH9; A0A175M4H1; A0A178LQP5; A0A179VE56; A0A1A0KPK7; A0A1A0L0U6; A0A1A0LPK1; A0A1A0MTI4; A0A1A0TFU8; A0A1A0U4I2; A0A1A0V5A9; A0A1A1WX30; A0A1A1ZXL5; A0A1A2D870; A0A1A2DTU2; A0A1A2H320; A0A1A2J0W0; A0A1A2LZ19; A0A1A2N2M9; A0A1A2NZG2; A0A1A2PC12; A0A1A2PHF9; A0A1A2SXD3; A0A1A2UY38; A0A1A2VD78; A0A1A2WGM9; A0A1A2WHS5; A0A1A2XKK7; A0A1A2YRA2; A0A1A2ZAZ4; A0A1A3BQQ7; A0A1A3C975; A0A1A3CCF6; A0A1A3DPL7; A0A1A3HDD0; A0A1A3HYY2; A0A1A3HZ67; A0A1A3JTU7; A0A1A3JVG7; A0A1A3MQE1; A0A1A3NWS4; A0A1A3PE10; A0A1A3QVL1; A0A1A6BFB6; A0A1B6BAV8; A0A1B9CSJ2; A0A1C3EPI5; A0A1C3HCR7; A0A1C5VPV3; A0A1C5Y6B5; A0A1C5ZLN1; A0A1C6A4W0; A0A1C6A6C8; A0A1C6A834; A0A1C6ADQ5; A0A1C6AG26; A0A1C6BPU9; A0A1C6BR67; A0A1C6F8H5; A0A1C6FJT7; A0A1C6FN12; A0A1C6KB52; A0A1C7G763; A0A1C7G7P7; A0A1D3N4S7; A0A1D7QC67; A0A1D7V1G2; A0A1E3RL64; A0A1E3SID3; A0A1E3SPW5; A0A1E4IAC6; A0A1E4M2X3; A0A1E8BNY6; A0A1F4G7Q6; A0A1F8LYX6; A0A1F8P3W3; A0A1F8PDB3; A0A1F8PND6; A0A1F8ZZD6; A0A1F9AET6; A0A1F9BAH3; A0A1G1HH93; A0A1G2YUL0; A0A1G4EJX1; A0A1G4PP51; A0A1G4WJ49; A0A1G5RWU8; A0A1G6A6P6; A0A1G6BY92; A0A1G7WGV6; A0A1G8SA93; A0A1G8TD42; A0A1G9QH12; A0A1H0T622; A0A1H2U2C5; A0A1H3BSE9; A0A1H4J3W2; A0A1H5AQE2; A0A1H5JY29; A0A1H5WYU5; A0A1H6URX3; A0A1H7C5B9; A0A1H7RW15; A0A1H7UKY5; A0A1H7Y7I6; A0A1I0IJ68; A0A1I0TSI6; A0A1I0UHA4; A0A1I0ZQG2; A0A1I0ZZD4; A0A1I1B8G0; A0A1I2UNL1; A0A1I3NQY4; A0A1I4NWY3; A0A115I6R5; A0A1I5KQT6; A0A1I7DV69; A0A1J0UBW8; A0A1K1LXH9; A0A1M5G8F1; A0A1M5HS05; A0A1M5WP08; A0A1M5Y2D8; A0A1M6CTW9; A0A1M6DNP4; A0A1M6EK40; A0A1M6RJT6; A0A1M6TZR5; A0A1M6WQT6; A0A1M7DB52; A0A1M7ERT5; A0A1M7YRH1; A0A1M7ZRY3; A0A1N6XVW6; A0A1N6YHD2; A0A1N7E6M3; A0A1N7ITG9; A0A1N7JA85; A0A1Q3GUI5; A0A1Q4ASN5; A0A1Q4BGR2; A0A1Q4HX34; A0A1Q6UWV9; A0A1Q9LMY7; A0A1Q9LPG2; A0A1Q9UGN3; A0A1R0UG01; A0A1R0VHU8; A0A1R3XZB6; A0A1S8FIM3; A0A1S8L514; A0A1S8LR37; A0A1S8LR40; A0A1S8T8Q5; A0A1S8T8S2; A0A1S8TNH6; A0A1S9CFW3; A0A1S9CT50; A0A1T4SYP6; A0A1T4SYT6; A0A1V2YH75; A0A1V2YIT4; A0A1V3X737; A0A1V3XES9; A0A1V6IPN5; A0A1V6ISS3; A0A1W1YWC9; A0A1W6EBJ9; A0A1W9Y150; A0A1X0A6W5; A0A1X0FC40; A0A1X0H7T8; A0A1X0IC14; A0A1X0J1P2; A0A1X0JIE2; A0A1X0KFV6; A0A1X0XZZ5; A0A1X1B9E3; A0A1X1R291; A0A1X1SSR7; A0A1X1TXR5; A0A1X1V3S4; A0A1X1VL32; A0A1X1W7E1; A0A1X1X7N0; A0A1X1XKW8; A0A1X1YLG5; A0A1X2AHF2; A0A1X2C2U1; A0A1X2CLH6; A0A1X2D9D7; A0A1X2DHC9; A0A1X2DSC7; A0A1X2LSD2; A0A1Y3LXZ6; A0A1Y5PDQ5; A0A1Y5TME4; A0A1ZINJZ2; A0A1Z4EIB2; A0A1Z9API7; A0A210WAI2; A0A212LML7; A0A239JG69; A0A239QDH3; A0A254T9L0; A0A255DPB2; A0A255Y6B9; A0A257DNX8; A0A257J691; A0A258BHL7; A0A258PB32; A0A258VG01; A0A259Y7J1; A0A259ZXU5; A0A260AVQ6; A0A260AZE6; A0A260IX48; A0A260MIL3; A0A260PYH9; A0A260RE70; A0A260SBS8; A0A260UD58; A0A260VNX6; A0A261D8D6; A0A263D9K5; A0A286ENR8; A0A286J393; A0A2A3GIA2; A0A2A4QQA2; A0A2A5JHH7; A0A2C8XDW0; A0A2C9T5A3; A0A2D6G1I1; A0A2D6G4K2; A0A2D6NZA9; A0A2D7DLF0; A0A2D7I0L2; A0A2D7PRZ6; A0A2D8IYT1; A0A2D8XJC3; A0A2D9TU36; A0A2E0EZ26; A0A2E0HBR0; A0A2E0IET8; A0A2E1ATH9; A0A2E1JHH9; A0A2E3BR16; A0A2E3CXI0; A0A2E4HQ67; A0A2E5VFH6; A0A2E6NWY9; A0A2E6YCU5; A0A2E8KTC2; A0A2E8RFV4; A0A2E9AP66; A0A2E9AVB3; A0A2E9GS86; A0A2E9J8Y6; A0A2E9W0B9; A0A2E9YIG6; A0A2G2D2W9; A0A2G3M789; A0A2G6RXD1; A0A2G6TXT1; A0A2G9WS52; A0A2I8DML4; A0A2J7YR92; A0A2K4YBB4; A0A2K8YQU1; A0A2K9LND0; A0A2M6VVB3; A0A2M8W6A7; A0A2M8Y9U6; A0A2M9Y3Z5; A0A2M9YSE5; A0A2N0AKE9; A0A2N0B3H6; A0A2N0BTI8; A0A2N0HNK3; A0A2N0XFZ3; A0A2N2TQX9; A0A2N3WW07; A0A2N7CY13; A0A2N7D7B9; A0A2N8PIB1; A0A2P2DEV3; A0A2R4BIK1; A0A2R5H6G3; A0A2R7LU49; A0A2S2BWE0; A0A2S4JCA3; A0A2S6A2T3; A0A2S8KT01; A0A2S8L495; A0A2S9FIX8; A0A2S9FJD5; A0A2S9FTW2; A0A2S9FTZ7; A0A2T2Y8S1; A0A2T2ZB26; A0A2T2ZH77; A0A2T7UT71; A0A2T8HTH4; A0A2U0SYZ2; A0A2U3N625; A0A2U3NZ03; A0A2U3PG11; A0A2V1JRE6; A0A2X1TET8; A0A2X2WVX6; A0A2X4UYM3; A0A2Y9C9Y4; A0A315S3U5; A0A317E5P2; A0A318H5L4; A0A318KF31; A0A318R8S9; A0A327X2K9; A0A327Z1R8; A0A329B8T7; A0A349SX03; A0A355T1S4; A0A356IPI1; A0A365XUQ3; A0A366GA73; A0A370CW33; A0A370D7V4; A0A377RVC1; A0A378RHB0; A0A378SDP6; A0A378TKT7; A0A378WVL1; A0A379G611; A0A379LRC3; A0A386U623; A0A388SE56; A0A396ZCQ5; A0A397N8V3; A0A3A1XZS0; A0A3A1Y6I7; A0A3A6IJA5; A0A3B8YSY6; A0A3B9AR55; A0A3B9XU39; A0A3B9YPW7; A0A3C1B0S7; A0A3C1B2K8; A0A3C1B2Z2; A0A3D0NM26; A0A3D1P2E2; A0A3D2S3N6; A0A3E0MVT7; A0A3F2V8F6; A0A3G2E8A2; A0A3G3GLN4; A0A3G3HKM3; A0A3G7A9P8; A0A3G9I2Q0; A0A3L8AUJ3; A0A3L8BEQ0; A0A3L8BHT0; A0A3L8C078; A0A3M0HQZ1; A0A3M3FFP1; A0A3M3KBM1; A0A3N0VLT6; A0A3N1QFB3; A0A3N2F0J7; A0A3N7AV85; A0A3N7D0W1; A0A3Q9KFB4; A0A3R6QNS5; A0A3R8S9I2; A0A3S0RLB2; A0A3S0U887; A0A3S4CRY1; A0A3S4IZR0; A0A3S4RXB1; A0A3S4YFQ4; A0A3T0TQS1; A0A3T0TQU8; A0A3T0ZVA2; A0A413WY35; A0A413WY37; A0A415E1N8; A0A415E3Z7; A0A415EJD1; A0A417VFN0; A0A418AC10; A0A418KY55; A0A418WJ11; A0A423FAT6; A0A424K2P6; A0A425W4Q5; A0A426D3N5; A0A431KRA5; A0A447GEJ3; A0A447Q666; A0A448LG69; A0A480AQP9; A0A494T9L6; A0A494YDK1; A0A495KB45; A0A496YZT5; A0A4D7QMG1; A0A4D7YTB6; A0A4P6FD82; A0A4P7XEY7; A0A4Q1S056; A0A4Q3NM33; A0A4Q3UIJ4; A0A4Q7ZAN5; A0A4Q9H273; A0A4Q9HKB4; A0A4R0KH60; A0A4R1FTE3; A0A4R1KZY9; A0A4R1QFG8; A0A4R1QMS0; A0A4R1QU74; A0A4R2HLI6; A0A4R2IBI5; A0A4R2LVJ8; A0A4R2LZJ1; A0A4R2M6Y7; A0A4R2MCG3; A0A4R2ZJ66; A0A4R3NHI7; A0A4R5WMK0; A0A4R5WTP2; A0A4R6KU71; A0A4R7UPB6; A0A4R8L0Y6; A0A4R8N0T4; A0A4R9G5G7; A0A4R9ITP0; A0A4R9J270; A0A4R9JBH6; A0A4R9JHY4; A0A4R9JRS8; A0A4R9JXS9; A0A4R9K990; A0A4R9LBJ0; A0A4R9LEL6; A0A4R9LXW1; A0A4S2H358; A0A4T0UVC8; A0A4T0V9G8; A0A4U0ZKR2; A0A4U1J511; A0A4U9V9S6; A0A4V2JHN3; A0A4V2PUG3; A0A4V2SGQ1; A0A4V6WMT5; A0A4Y8Z2D9; A0A4Y9ET38; A0A4Z0NAS7; A0A4Z0ZRA5; A0A4Z1A5S8; A0A511QVB6; A0A515A4Z2; A0A515FVH0; A0A515YEM0; A0A519G2S2; A0A519I680; A0A519YWS8; A0A519ZMA4; A0A520FG23; A0A520THB4; A0A520TRN4; A0A520UA43; A0A520WX76; A0A520XU23; A0A520YRD2; A0A521DSC9; A0A521G7E6; A0A521Z340; A0A521ZRN4; A0A523SGX5; A0A542C1F7; A0A542V3D8; A0A543AKZ1; A0A547BKH8; A0A553A7W5; A0A561BEE6; A0A562PK97; A0A5B1BMZ2; A0A5C6WNU7; A0A5C7MDT3; A0A5C7XCF9; A0A5E8HC52; A0A5F1YD84; A0A5F1ZR95; A0A5F1ZTL5; A0A5F2ABL1; A0A5F2B813; A0A5F2BFY3; A0A5F2CJS5; A0A5F2D446; A0A5F2DEV9; A0A5F2EBM4; A0A5M3VBH7; A0A5N0V886; A0A5P3A223; A0A5R8WNP8; A0A5R9QRH4; A0A5S3W6T5; A0A5S3W706; A0A5S9BTY9; A0A5S9Q2D1; A0A643FL96; A0A653ECQ3; A0A653FFI9; A0A654TX64; A0A655FMN7; A0A655JJD9; A0A660NHY9; A0A679GQD0; A0A6A7YAM0; A0A6D2ACN0; A0A6G2TVR9; A0A6G2TWG5; A0A6G3D3V4; A0A6G8IKD1; A0A6H1LFF9; A0A6H1NCL7; A0A6H1P4G4; A0A6H1WWM0; A0A6H3NJ49; A0A6H3NXR3; A0A6I2Y8M6; A0A6I3MB43; A0A6I3ZMM3; A0A6I7HNN4; A0A6J4E5H3; A0A6J5AP79; A0A6J5ITD5; A0A6J5IV47; A0A6L6R5M2; A0A6L6VJX6; A0A6N4QBC2; A0A6N4QD72; A0A6N4V467; A0A6N4WIG2; A0A6N6VLF0; A0A6N7KWS9; A0A6N7YLQ4; A0A6N9HZQ4; A0A6P2GRS8; A0A6P2Y5Y4; A0A6S4TF48; A0A6S5AEC2; A0A6S5ILS1; A0A6S5RV84; A0A6S6MRV5; A0A6S7AT12; A0A6S7DJN6; A0A6S7E996; A0A6S7ESJ1; A0A7C2IKP2; A0A7C2T351; A0A7C3GGX0; A0A7C3KLG2; A0A7C3T4C9; A0A7C3X0Y3; A0A7C7JF16; A0A7C7U9C7; A0A7C7WM30; A0A7C7YAN8; A0A7C7ZKG1; A0A7C9TL90; A0A7G1IB95; A0A7G1KKZ2; A0A7G1L1R0; A0A7G2JLR9; A0A7G2TYY4; A0A7G8PH87; A0A7H4UIH6; A0A7H5K0S8; A0A7I0HMF6; A0A7I6PZI9; A0A7I6V7I3; A0A7I7KPY8; A0A7I7LN49; A0A7I7M5N0; A0A7I7MPQ8; A0A7I7N2Q9; A0A7I7NWL5; A0A7I7Q2L2; A0A7I7SRA4; A0A7I7T348; A0A7I7U0E2; A0A7I7WUJ0; A0A7I7YQN7; A0A7I7ZTN1; A0A7I9WIC9; A0A7I9YQ92; A0A7K0LMR3; A0A7K0P1K9; A0A7K0Q2N9; A0A7K0RKR6; A0A7K1JGL3; A0A7K3QSK2; A0A7K3QSU4; A0A7T7UYS4; A0A7U3Z1B3; A0A7U4FB36; A0A7U6KD82; A0A7U6LHN4; A0A7U6QHV1; A0A7U9E5M2; A0A7U9Q8W1; A0A7V2W349; A0A7V4N9X6; A0A7V6ZIN1; A0A7W5BF73; A0A7W5BFF5; A0A7W5ZQB6; A0A7W6J5Z7; A0A7W8EJ44; A0A7W8HA28; A0A7W8HAI5; A0A7W8HAP1; A0A7W9UFJ5; A0A7W9WMP6; A0A7W9ZF36; A0A7X0ITT3; A0A7X0J0L1; A0A7X5TUM0; A0A7X6FMU7; A0A7X6JDH6; A0A7X6L6E7; A0A7X6PB96; A0A7X8JHY4; A0A7X9PHX4; A0A7X9VSI0; A0A7Y1U756; A0A7Y1ZIU3; A0A7Y3BQI5; A0A7Y3DCM5; A0A7Y4H1L7; A0A7Y5J669; A0A7Y9H556; A0A806JT16; A0A822VAQ6; A0A822VC44; A0A829CBP1; A0A829QZL6; A0A833HAZ4; A0A837R9T3; A0A839RPV8; A0A844BBU6; A0A846FCI0; A0A846VWL3; A0A847DA30; A0A847EIY0; A0A848GMY3; A0A848KWE3; A0A848V0Y3; A0A848VCT7; A0A848ZGD9; A0A849G5R2; A0A850PTQ5; A0A850SNV5; A0A853SBD0; A0A856NFJ9; A0A857KQ80; A0A857M6B8; A0A8B3CUF4; A0A8B4ARF4; A0A8B4DTJ8; A0A8B4GLP4; A0A8B4YHY7; A0A8D6KZE3; A0A8D6ME42; A0A8E3AIK2; A0A8E4KT51; A0A8F9WRB3; A0A8G1TSD4; A0A8G2LD11; A0A8H2PEU6; A0A8I0JX45; A0A8I1P2G6; A0A8J6MAB6; A0A8J7J2R2; A0A8S0HAH4; A0A8T7KQH8; A0KN52; A0QV85; A1T1F8; A4SK01; A4T3L6; A5U3E4; A9C202; B0SJ17; B4SIW7; B6XKA0; B8FHD0; B8KKX0; B8L3N1; C5EGJ9; C6LBF7; C6LBF9; D1P482; D1P483; D2BZW0; D2RJU0; D3ANW9; D5P3M3; E6TJT7; E7RUF7; F6B214; F7T8L2; F7V2N6; F7V4C6; F7V5F7; F7V732; F7V7J7; F7V872; G0TKQ7; G4HYG9; G7GMB9; G8LP40; G8RIY5; G9YME3; H0E5I4; H0SPF1; H1CE97; H1D1Q2; H3NX20; I0HKF6; I0HNL8; I4B3M5; I7G056; I9AIY1; J1SC70; J4PFL3; J7U848; J7VLE6; J8BQM8; J8CZN8; J8F6R6; K1KDY6; K8XJP1; L7N5M3; L7VU82; L8DMN7; M0QFR6; M1MMS1; M1N4Z2; M1PNJ4; M5CRE7; M5D328; M7CE42; N1VRW8; N1WHN2; 033178; Q2RWL1; Q7NWZ1; R4M6B1; R5T9I1; R5TF53; R5THE8; R5TSF7; R7M1T0; R8DG38; R8W5W3; R9A1Z9; T1XI02; T2IMR9; T2K0G8; U2PFP1; U2QRP6; U2RJ59; U5WIR8; U6ZWT8; U6ZXB9; V8D3J9; W5THL3; X5MFC8; X7XY78; X7ZF09.

Exemplary lipase enzymes suitable for the processes of the present disclosure includes, but not limited to, triacylglycerol lipase (EC No. 3.1.1.3), acylglycerol lipase (EC No. 3.1.1.23), and sn-1-specific diacylglycerol lipase (EC No. 3.1.1.116). Protein sequences corresponding to the lipase enzymes are well known in the art and the same can be used in the processes of the present disclosure. Exemplary UNIPROT sequence IDs for the processes of the present disclosure include, but not limited to, the following:

| | UNIPROT |
|---|---|
| Name | ID |
| Probable secreted lipase ARB_02369 (EC 3.1.1.3) | D4B1N9 |
| Secreted lipase ARB_01498 (EC 3.1.1.3) | D4AZ78 |
| Secreted lipase ARB07186/07185 (EC 3.1.1.3) | D4ASH1 |
| Triacylglycerol lipase 1 (EC 3.1.1.3) | Q71DJ5 |
| Triacylglycerol lipase 3 (EC 3.1.1.3) (Lipase 3) | P40308 |
| Triacylglycerol lipase 4 (EC 3.1.1.3) (Lipase 4) | P36165 |
| Triacylglycerol lipase 5 (TAG lipase 5) | Q12043 |
| (TG lipase 5) (EC 3.1.1.3) (Lipase 5) | |
| Triacylglycerol lipase OBL1 (EC 3.1.1.—) | F4JFU8 |
| (Oil body lipase 1) (AtOBL1) | |
| Triacylglycerol lipase OBLI (EC 3.1.1.—) | A0A1S3ZP85 |
| (Oil body lipase 1) (NtOBL1) | |
| Triacylglycerol lipase ptl1 (EC 3.1.1.3) | Q9Y7P3 |
| Triacylglycerol lipase ptl2 (EC 3.1.1.3) | O14115 |
| Triacylglycerol lipase ptl3 (EC 3.1.1.3) | Q9Y827 |
| Triacylglycerol lipase SDP1 (EC 3.1.1.3) | Q9LZA6 |
| (Protein SUGAR-DEPENDENT 1) | |
| Triacylglycerol lipase SDP1L (EC 3.1.1.3) | Q9M1I6 |
| (Protein SDP1-LIKE) | |

The processes of the present disclosure can be effected in a batch mode. Alternatively, the processes of the present disclosure can be effected in a fed batch mode. Still alternatively, the processes of the present disclosure can be effected in a continuous mode.

The process can be effected at various scales ranging from 1-10,000,000 liter scale. Batch mode reactors are run at the largest volumes. Batch reactions are run with polyphenol, aqueous medium, and enzyme loaded into the reactor at process initiation. Fed batch reactors have higher per-unit volume productivity as reactant and enzyme may be fed into a batch tank, until process completion. Continuous flow reactors have the volumes due to their high per-unit volume productivity.

In some embodiments, the enzyme is immobilized onto a suitable carrier substrate, such as a resin substrate. Exemplary resins suitable for the processes of the present disclosure include, but not limited to, phenol-formaldehyde, agarose, Sepharose, silica, polyvinyl alcohol, vinyl sulfone, cellulose, acrylate ester, polystyrene, and mixtures thereof. Alternatively, the enzyme can be dissolved or otherwise dispersed in a suitable vehicle, such as an aqueous medium.

In some embodiments, free enzyme is recycled from the aqueous medium by filtration, for efficient recycling.

The step of contacting the polyphenol with the enzyme can be effected at a temperature ranging from about 4° C. to about 150° C., for example, from about 10° C. to 120° C., or from about 25° C. to about 100° C., or from about 30° C. to 80° C. In some embodiments, the step of contacting the polyphenol with the enzyme is effected at a temperature ranging from about 25° C. to about 100° C., for example, from about 30° C. to about 90° C., or from about 30° C. to about 80° C., or from about 30° C. to about 70° C.

The step of contacting the polyphenol with the enzyme can be effected for a time period ranging from about 1 second to about 72 hours, for example, from about 30 minutes to about 40 hours or from about 45 minutes to about 30 hours or from about 60 minutes to about 25 hours or from about 2 hours to about 10 hours. In some embodiments, the step of contacting the polyphenol with the enzyme is effected for a time period ranging from about 30 minutes to about 30 hours, for example, from about 45 minutes to about 20 hours or from about 1 hour to about 15 hours or from about 2 hours to about 10 hours or from about 2 hours to about 7 hours.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the scope of the embodiments as described herein.

### EXAMPLES

### Enzyme Screening

Enzymes shown in Table 1 were screened for naringenin, hesperetin, and chalcone hydrolysis. All the enzymes converted the polyphenols to phloroglucinol and cinnamic acids (phenolic derivative) at millimolar concentrations as proven by HPLC traces.

TABLE 1

| Organism | Protein ID | Protein Name |
|---|---|---|
| Eubacterium | Q715L4 | Phloretin Hydrolase |
| ramulus | | |
| Lactiplanti- | WP_152706034.1 | Phloretin Hydrolase |
| bacillus | | |
| pentosus | | |
| Pseudomonas | MBP5150568.1 | MBP5150568.1 2,4- |
| protegens | | diacetylphloroglucinol |
| | | hydrolase |
| Emergencia | WP_067539625.1 | Phloretin Hydrolase |
| timonensis | | |

| Protein ID | Enzyme Sequences |
|---|---|
| Q715L4 | >sp/Q715L4.1\|PHY_EUBRA RecName: Full = |
| | Phloretin hydrolase |
| | MEEDFNMSTPGVKVGVXEEEKKLSYYKYYEQDLAPVPAEKIAI |
| | LQGGPIAPEKCIPFDERNKFLKGEDDEYANIGFGVAADGTALV |
| | CNTTYMPGVTGEMLDWWFPWHSVGSDLRYKIWDPEDHYFARAY |
| | PASYVVDPNVPMNQKTWGVDHYIMEDVGPGPEFLKLCFKRPAD |
| | FGYDESIIGTEKCESLVCAIGESSCAAAMTHKWHPYKDGVLFE |
| | SRFWIGYRIDEEGNIVKAIPEGVSIPPFVPQGLFAHNIKEFTN |
| | LAAILPTLYAEEKDTF |
| WP_152706034.1 | >WP_152706034.1 phloretin hydrolase |
| | [Lactiplantibacillus pentosus] |
| | MTEKFTQNIPANAATLPYYKYYLQDMAKVDPTARKKMVQAPID |
| | PKDATPVQDRNDLLKPGYLKTEVGYCQMPDGTAFMANHLKMPN |
| | VTAEMLHWWFAWHGLESMRYVIWNKDDHYDVVVKNGTEAQLKD |
| | QSISMAERIYGVSHTVTEDTGFGPEKIDINFKNPVDLGYDPQL |
| | LAQSDSDIVAAANGETALMLHVVRPVDDGIELRSRFWLGWNVD |
| | LQTHEPVRVIPDDAKIDGEVAKRLGLHNIKEMTNLAQILPSLY |
| | AENKDKF |
| MBP5150568.1 | >MBP5150568.1 2,4-diacetylphloroglucinol |
| | hydrolase, partial [Pseudomonas protegens] |
| | MEARNMTPFTYFSLPMQKLFLRNQAAVRNKPYAKYFRSEMRVP |
| | LSAVRKIQQGPMALEDTLTPSIEDINRLLEPDFVSEESGYALL |
| | PGPMAYVQSRKFFPGCTAQMFKWWFIWHPAESERYTLWFPYAH |
| | VSNPCVHHQRLCDESLSFEERLYGNTFCASEYVGDRLMHLHID |
| | FQQPASLGLNTDLY |
| WP_067539625.1 | >WP_067539625.1 phloretin hydrolase |
| | [Emergencia timonensis] |
| | MSQRPVLTAEERKLPYAKYYDLPITPIPAEKIAVLEAGPIDPS |
| | LALKIEDRNKLFEPGYLPCEIGYCVMEDGSAYLANRTEMPGVT |
| | PEMFEWWFAWHGLEDMRYRIWDPEDHFYARQQMREKVLDPKVP |
| | MREKTWGTVHIVREDIGAGPDDLILEFRYPHELGYDESKVGTK |
| | DCAAMMCANGHGPVPGQGVAAVMTHMVREIEGGIELRSRFWIG |
| | WGLVNGQVVKLVPDGVRVPVEVPMGLFAHNLKEFGHLATILPD |
| | LYKEEKDHF |

### Colorimetric Enzyme Screening Assay

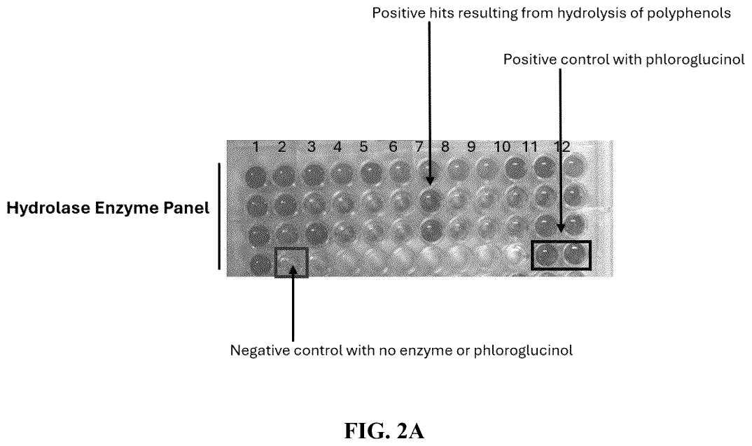

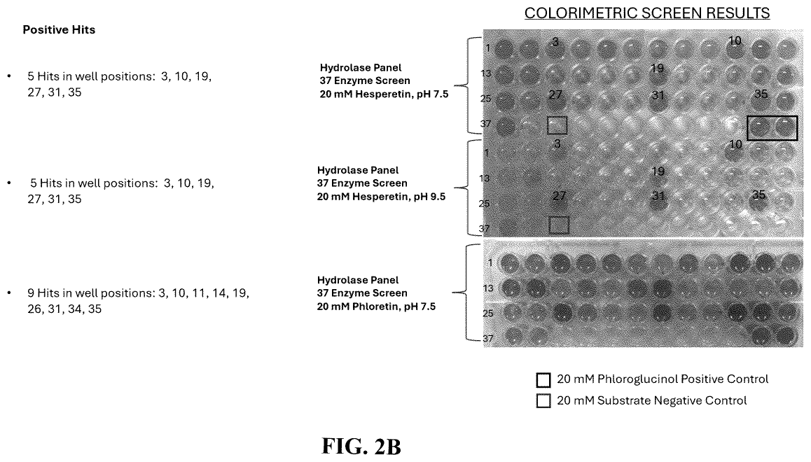

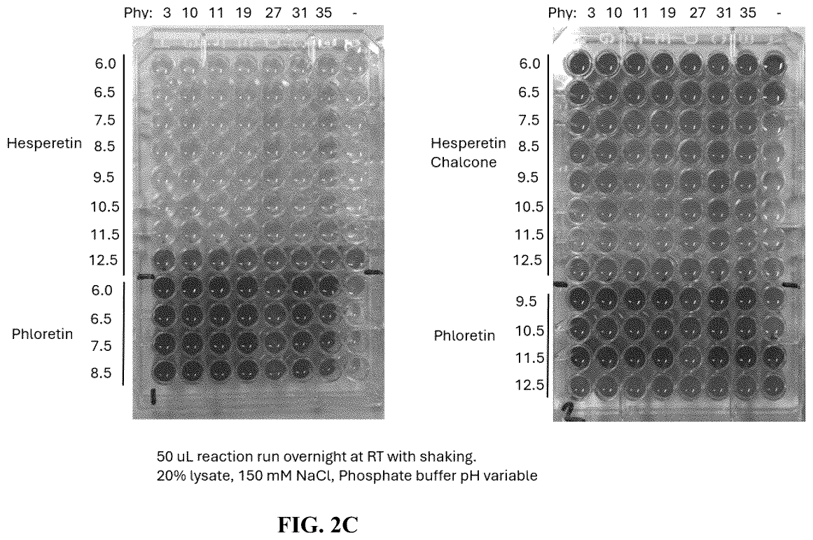

Enzyme activity was measured using colorimetric assay. We have a colorimetric readout (FIG. 2A), where orange coloration is evidence of phloroglucinol formation. Darker orange coloration implies higher phloroglucinol concentration. The colorimetric assay is as described in Chatterjee & Gibbins, 1969 uses vanillin and HCl to create a highly specific readout for phloroglucinol. We followed their protocols to quantify. FIGS. 2B and 2C show the results of a 37-enzyme screening panel, with the colorimetric confirmation of hydrolysis of both hesperetin and hesperetin chalcone.

### Enzymatic Hydrolysis Reaction Converting Hesperetin Chalcone to Phloroglucinol and Isoferulic Acid

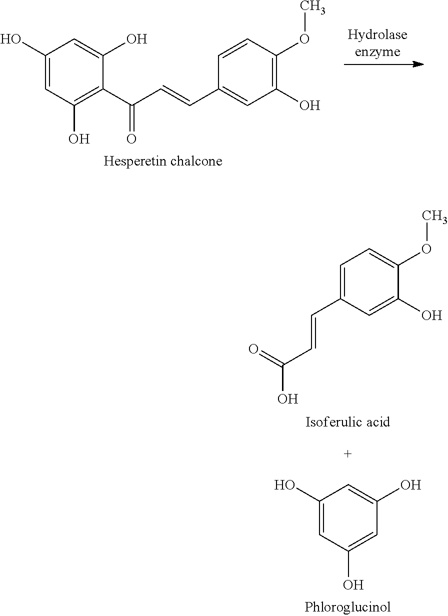 [SMILES: COc1ccc(/C=C/C(=O)c2c(O)cc(O)cc2O)cc1O ; COc1ccc(/C=C/C(=O)O)cc1O ; Oc1cc(O)cc(O)c1]

Hesperetin chalcone (20 mM final conc.) was loaded to a 1.5 mL tube containing 150 mM NaCl, 50 mM NaHPO₄, and 10% by volume bacterial lysate. The reaction was incubated for 16 hrs at 25° C. The resulting products were analyzed by HPLC to determine % conversion, phloroglucinol production, and production of isoferulic acid. Products were separated by HPLC using water/acetonitrile binary gradient and yield was determined by HPLC of peak size relative to standards.

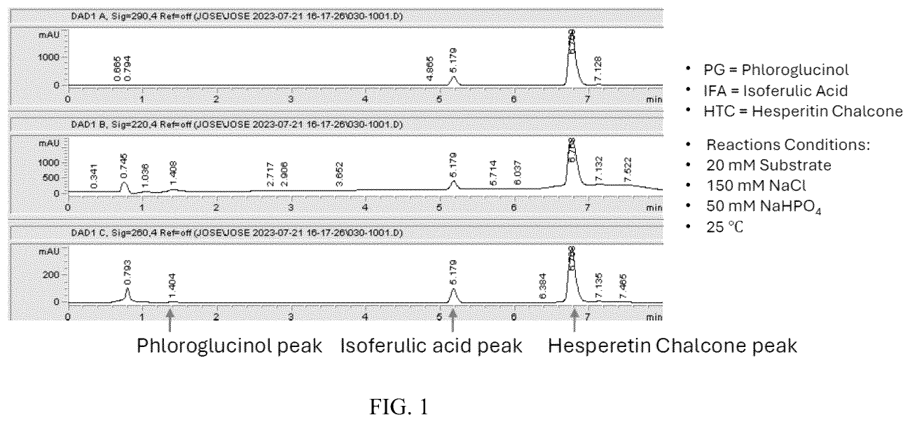

In other variations the procedure above was repeated, but with purified enzymes. In other variations the enzyme was left in the cell and used as whole cell.
Hydrolase enzymes were expressed in E. coli BL21(DE3) with pET28 vector under control of a T7 promoter.
In other variations the enzymes were constitutively expressed in E Coli. Other variations used a genome integrated copy of the hydrolase enzyme.
FIG. 1 shows HPLC analysis results, demonstrating that hesperetin chalcone is converted to phloroglucinol and isoferulic acid by the hydrolase enzyme.

### Enzymatic Hydrolysis Reaction Converting Hesperetin to Phloroglucinol and Isoferulic Acid

Hesperetin chalcone was generated by in situ conversion from hesperetin by the following protocol. 100 milligrams hesperetin was loaded to a 50 mL round bottom flask in 10 mL distilled water. 10M NaOH solution was added dropwise to the solution while mixing until pH 14 was reached. The solution was incubated at room temperature while stirring for 1 hour. A 100 mL solution of 1M HCl was cooled on ice, then alkaline hesperetin solution was added dropwise under vigorous mixing. The resulting product was filtered and washed with deionized water to obtain high purity hesperetin chalcone. The resulting chalcone was analyzed by HPLC and found to be primarily hesperetin chalcone with approximately 15% residual hesperetin. The hesperetin chalcone was used as previously described to yield phloroglucinol and isoferulic acid by enzymatic hydrolysis.

1. A process for production of phloroglucinol or a derivative thereof, comprising contacting a polyphenol with a hydrolase enzyme.

2. The process of claim 1, wherein the polyphenol is selected from flavonoid aglycones, flavonoid glycosides, chalcones, and mixtures thereof.

3. The process of claim 1, wherein the polyphenol is selected from naringin, naringenin, hesperidin, hesperetin, eriodictyol, homoeriodictyol, rutin, dihydrokaempferol, dihydroquercetin, naringenin chalcone, liquiritigenin, afzelechin, mesquitol, chalcone, echinatin, hesperidin chalcone, hesperetin chalcone, naringin chalcone, and mixtures thereof.

4. The process of claim 1, wherein the polyphenol is represented by Formula A or Formula B, 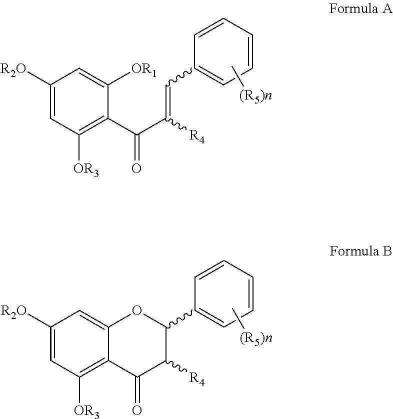
wherein R₁, R₂ and R₃ are independently selected from H, C1-C4 alkyl group and a sugar moiety; R₄ is selected from H, OH and —OR₆, R₆ being a sugar moiety; R₅ is selected from OH, C1-C4 alkyl group and —OR₇, R₇ being a C1-C4 alkyl group or a sugar moiety; and n ranges from 0-5.

5. The process of claim 4, wherein the polyphenol is represented by Formula A, and is selected from hesperidin chalcone, hesperetin chalcone, naringin chalcone and mixtures thereof.

6. The process of claim 4, wherein the polyphenol is represented by Formula B, and is selected from naringin, naringenin, hesperidin, hesperetin, eriodictyol, homoeriodictyol, rutin, dihydrokaempferol, dihydroquercetin and mixtures thereof.

7. The process of claim 1, wherein the hydrolase enzyme is selected from 2,6-dihydroxypseudooxynicotine hydrolase (EC No. 3.7.1.19), 2,4-diacetylphloroglucinol hydrolase (EC No. 3.7.1.24), phloretin hydrolase (EC No. 3.7.1.4), 2,6-dioxo-6-phenylhexa-3-enoate hydrolase (EC No. 3.7.1.8), triacylglycerol lipase (EC No. 3.1.1.3), acylglycerol lipase (EC No. 3.1.1.23), and sn-1-specific diacylglycerol lipase (EC No. 3.1.1.116).

8. The process of claim 1, wherein the step of contacting the polyphenol with the hydrolase enzyme is performed at a basic pH.

9. The process of claim 8, wherein the basic pH is obtained by addition of a base.

10. The process of claim 9, wherein the base is selected from sodium hydroxide, potassium hydroxide, potassium carbonate, sodium carbonate, lithium hydroxide, calcium hydroxide, ammonium hydroxide, pyridine, triethylamine, trimethylamine, aniline, urea, aluminum hydroxide, sodium bicarbonate, and mixtures thereof.

11. The process of claim 1, wherein the polyphenol is contacted with the hydrolase enzyme in presence of a buffer.

12. The process of claim 11, wherein the buffer is selected from salts of phosphate, carbonate, tricine, bicine, hepes, 3-morpholinopropane-1-sulfonic acid (MOPS), 2-(N-morpholino)ethanesulfonic acid (MES), N-[Tris(hydroxymethyl)methyl]-2-aminoethanesulfonic acid (TES), 3-[Tris-(hydroxymethyl)Methylamino]-2-Hydroxypropane Sulphonic Acid (TAPSO), glycine, lysine, acetate, borate, and mixtures thereof.

13. The process of claim 11, wherein said buffer is used in a concentration ranging from about 1 mM to about 1000 mM.

14. The process of claim 1, wherein the step of contacting the polyphenol with the hydrolase enzyme is effected at a temperature ranging from about 25° C. to about 100° C.

15. The process of claim 1, wherein the step of contacting the polyphenol with the hydrolase enzyme is effected for a time period ranging from about 30 minutes to about 30 hours.

16. A process for production of a phenolic derivative of Formula C 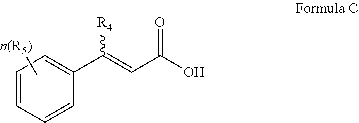
wherein R₄ is selected from H, OH and —OR₆, R₆ being a sugar moiety; and R₅ is selected from OH, C1-C4 alkyl group and —OR₇, R₇ being a C1-C4 alkyl group or a sugar moiety; and n ranges from 0-5;
comprising contacting a polyphenol of Formula A or Formula B 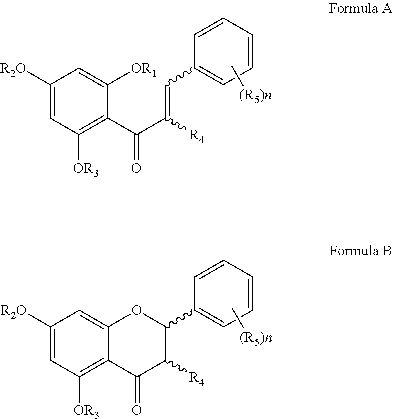
wherein R₁, R₂ and R₃ are independently selected from H, C1-C4 alkyl group and a sugar moiety; R₄ is selected from H, OH and —OR₆, R₆ being a sugar moiety; R₅ is selected from OH, C1-C4 alkyl group and —OR₇, R₇ being a C1-C4 alkyl group or a sugar moiety; and n ranges from 0-5.
with a hydrolase enzyme.

17. The process of claim 16, wherein the polyphenol is represented by Formula A, and is selected from hesperidin chalcone, hesperetin chalcone, naringin chalcone and mixtures thereof.

18. The process of claim 16, wherein the polyphenol is represented by Formula B, and is selected from naringin, naringenin, hesperidin, hesperetin, eriodictyol, homoeriodictyol, rutin, dihydrokaempferol, dihydroquercetin and mixtures thereof.

19. The process of claim 16, wherein the hydrolase enzyme is selected from 2,6-dihydroxypseudooxynicotine hydrolase (EC No. 3.7.1.19), 2,4-diacetylphloroglucinol hydrolase (EC No. 3.7.1.24), phloretin hydrolase (EC No. 3.7.1.4), 2,6-dioxo-6-phenylhexa-3-enoate hydrolase (EC No. 3.7.1.8), triacylglycerol lipase (EC No. 3.1.1.3), acylglycerol lipase (EC No. 3.1.1.23), and sn-1-specific diacylglycerol lipase (EC No. 3.1.1.116).

20. The process of claim 16, wherein the step of contacting the polyphenol of Formula A or Formula B with the hydrolase enzyme is performed at a basic pH.

21. The process of claim 20, wherein the basic pH is obtained by addition of a base.

22. The process of claim 21, wherein the base is selected from sodium hydroxide, potassium hydroxide, potassium carbonate, sodium carbonate, lithium hydroxide, calcium hydroxide, ammonium hydroxide, pyridine, triethylamine, trimethylamine, aniline, urea, aluminum hydroxide, sodium bicarbonate, and mixtures thereof.

23. The process of claim 16, wherein the polyphenol of Formula A or Formula B is contacted with the hydrolase enzyme in presence of a buffer.

24. The process of claim 23, wherein the buffer is selected from salts of phosphate, carbonate, tricine, bicine, hepes, 3-morpholinopropane-1-sulfonic acid (MOPS), 2-(N-morpholino)ethanesulfonic acid (MES), N-[Tris(hydroxymethyl)methyl]-2-aminoethanesulfonic acid (TES), 3-[Tris-(hydroxymethyl)Methylamino]-2-Hydroxypropane Sulphonic Acid (TAPSO), glycine, lysine, acetate, borate, and mixtures thereof.

25. The process of claim 23, wherein said buffer is used in a concentration ranging from about 1 mM to about 1000 mM.

26. The process of claim 16, wherein the step of contacting the polyphenol of Formula A or Formula B with the hydrolase enzyme is effected at a temperature ranging from about 25° C. to about 100° C.

27. The process of claim 16, wherein the step of contacting the polyphenol of Formula A or Formula B with the hydrolase enzyme is effected for a time period ranging from about 30 minutes to about 30 hours.
